(19) United States
(12) Patent Application Publication
(10) Pub. No.: US 2011/0258915 A1
(43) Pub. Date: Oct. 27, 2011

(54) Method and Unit for Large-Scale Algal Biomass Production

(75) Inventor: Bobban Subhadra, Albuquerque, NM (US)

(73) Assignee: STC.UNM, Albuquerque, NM (US)

(21) Appl. No.: 13/124,631

(22) PCT Filed: Oct. 19, 2009
(86) PCT No.: PCT/US09/61127

§ 371 Date: Jun. 22, 2011

Related U.S. Application Data
(60) Provisional application No. 61/106,222, filed on Oct. 17, 2008.
(60) Provisional application No. 61/106,218, filed on Oct. 17, 2008.

(51) Int. Cl. C10L 1/188; A01G 1/00; C07C 29/88; B01D 35/02; C12M 1/12; C07K 1/14

(52) U.S. Cl. 44/385; 435/297.2; 530/412; 568/854; 210/407; 47/1.4

(57) ABSTRACT

Methods and apparatus for growing and sequentially processing algae to produce a variety of products including but not limited to biofuels, algal meal, oil, unsaturated fatty acids and recombinant proteins and peptides in a cost-effective and energy efficient manner. Also provided are methods of use for the products.

### CROSS-REFERENCE TO RELATED APPLICATIONS

The following application claims benefit of U.S. Provisional Application Nos. 61/106,218 and 61/106,222 both filed Oct. 17, 2009 and both of which are hereby incorporated by reference in their entirety.

### BACKGROUND

Photosynthetic algae naturally produce many valuable health-enhancing products such as highly unsaturated fatty acids and carotenoids. Recent genetic engineering studies have also shown that algae offer the potential to produce high yields of recombinant proteins more rapidly and at much lower cost than traditional cell culture. Extensive research was conducted to determine the utilization of microalgae as an energy source, with applications being developed for biodiesel, ethanol, and hydrogen gas production. Because of the multiple uses of algae, there is a growing interest in the large-scale (high density) controlled-environment production of algae from different industries. Known methods of controlled-environment production of algae include pond production and bioreactors.

Open ponds can be an important and cost-effective component of large-scale cultivation technology, and optimal design parameters have been known for many years. The elongated raceway-type of open pond, using paddlewheels for recirculation and mixing with an optimal depth of approximately 10-20 cm has been shown to be good for algal culture (Jassby 1988). A critical parameter for pond production is light penetration, which clearly is dependent on cell concentration. Unfortunately, traditional pond production systems have contamination problems and full automation of very large-scale operations is very difficult. In the past, large scale attempts to cultivate H. pluvialis in open ponds has met with failure (Bubrick 1991).

Closed photobioreactors have been used or proposed for the culture of microalgae. The most common are vertical or horizontal tubular or thin panel photobioreactors. The critical design requirement is to supply light efficiently by maximizing the illumination surface-to-volume ratio of the reaction. The method has several advantages: the risk of contamination is severely reduced and environmental parameters such as temperature and light can be controlled. When using a tubular design photobioreactor in a green house, over 500 g of dry algae per m3 per day can be produced. Unfortunately, while high biomass production can be attained in tubular and other designs, the high level of biomass production required by many applications is not reproducible when translated to large-scale operations.

Clearly, both existing methods have limitations when it comes to large-scale production of algal biomass. In addition to the problems identified above, both pond and bioreactor methods require a significant amount of external energy input, which increases the production cost of large-scale operations. Finally, both traditional pond production systems and bioreactors suffer due to the significant land-use requirements created by the demand for algal products. For example, current methods of algal-based biofuel production may require very large-scale operations extending thousands of hectors of land. Neither the pond production nor bioreactor methods have been demonstrated to be practical for this size of an operation.

Accordingly, novel systems for large-scale production of algae are required.

### BRIEF DESCRIPTION OF THE DRAWINGS

FIG. 1 is a schematic illustration of an exemplary Terraced Wall Algal Growth (TWAG) unit according to an embodiment of the invention.

FIG. 2 is a schematic side view of a TWAG array comprising a common collection chamber.

FIG. 3 is a schematic overhead view of 48 individual TWAG units arrayed in two separate green houses.

FIG. 4 is a schematic side view of an exemplary algal filtration unit.

FIG. 5 is a schematic top view of the exemplary algal filtration unit of FIG. 4.

FIG. 6 depicts the algal filtration unit of FIG. 4 in action as algae is pumped into the unit.

FIG. 7 depicts the algal filtration unit of FIG. 4 during vacuum filtration.

FIG. 8 depicts the algal filtration unit of FIG. 4 after vacuum filtration.

FIG. 9 depicts the algal filtration unit of FIG. 4 after the sieve unit has been rotated 90 degrees.

FIG. 10 depicts the algal filtration unit of FIG. 4 after the sieve unit has been rotated 180 degrees.

FIG. 11 depicts the algal filtration unit of FIG. 4 during high jet sieve washing.

FIG. 12 is a flowchart of an exemplary sequential processing method according to an embodiment of the present disclosure.

FIG. 13 is a schematic representation of an exemplary facility configured to grow and sequentially process algal biomass.

FIG. 14 is a flowchart showing another exemplary sequential processing method according to the present disclosure.

FIG. 15 is a block flow diagram showing yet another embodiment of a sequential process according to the present disclosure.

FIG. 16 is a flowchart showing exemplary production yields when practicing the methods described herein.

FIG. 17 is a schematic illustration of the ways in which renewable energy sources can be utilized in combination with the methods and apparatus of the present disclosure.

### DETAILED DESCRIPTION

According to an embodiment, the present disclosure provides a Terraced Wall Algal Growth (TWAG) unit for the controlled environment high density production of algal biomass which can be used for the production of highly unsaturated fatty acids, beta-carotene, recombinant proteins and biofuels (such as biodiesel, bioethanol and hydrogen gas). Operational steps include inoculation, growth phase-1, growth phase-2 (stress-shock to increase lipid content), harvesting, and re-inoculation. The design of the TWAG unit is optimized to provide maximum surface area and also for the automation of the entire production operations.

Algae are a large and diverse group of simple, typically autotrophic organisms, ranging from unicellular to multicellular forms. Though the prokaryotic cyanobacteria (commonly referred to as blue-green algae) were traditionally included as “algae” in older textbooks, many modern sources regard this as outdated and restrict the term algae to eukaryotic organisms. By modern definitions algae are eukaryotes and conduct photosynthesis within membrane-bound organelles called chloroplasts. Chloroplasts contain circular DNA and are similar in structure to cyanobacteria, presumably representing reduced cyanobacterial endosymbionts. Algae can live in almost any biological niche ranging from extreme psychroplic deep sea oceans to fresh water hot springs. Most of the simpler algae are unicellular flagellates or amoeboids, but colonial and non-motile forms have developed independently among several of the groups.

Since time immemorial, human beings used different species of algae for multiple purposes. The fast growth, highly nutritious constituents and bioactive biochemical content of many algal species has attracted many industries.

Algae used as edible food are generally marine and are commonly termed as ‘seaweeds’. The major users of seaweeds as food are the coastal Japanese, and about 25% of their daily diet consists of seaweeds. Seaweeds are used as food in many forms in several Asian countries, such as Myanmar, China, Thailand, Korea, Malaysia, Philippines and Indonesia, and are also considered a tasteful dish in England and Scotland. New Zealand, France, Chile, Hawaii, Brazil and several other Latin American countries also have edible algae based diets. About a hundred species of algae are used as food throughout the world, most of them belonging to the classes Chlorophyceae (green algae), Rhodophyceae (red algae) and Phaeophyceae (brown algae). Species of the genera Caulerpa, Durvillea, Laminaria, Monostroma, Nereocytstis, Oedogonium Nereocytstis, Oedogonium, Porphyra, Rhodymenia, Sargassum, and Spirogyra are particularly commonly used as food in different parts of the world. Seaweeds are not just alternate and exotic sources of food, but they also possess great nutritional value. They are rich in proteins, fats, vitamins, and mineral salts. Typically, the total dry weight of algae consist of about 25-30% fats; 10-20% proteins; 2-4% vitamins and 0.2-0.5% mineral salts. About a hundred species of algae are used as food throughout the world, most of them belonging to the classes Chlorophyceae (green algae), Rhodophyceae (red algae) and Phaeophyceae (brown algae).

Microalgae are employed in aquaculture as live feeds for all growth stages of bivalve molluscs (eg. oysters, scallops, clams and mussels), for the larval/early juvenile stages of abalone, crustaceans, and some fish species, and for zooplankton used in aquaculture food chains. Several hundred microalgal species have been tested as food, but probably less than twenty have gained widespread use in aquaculture. Microalgae must possess a number of key attributes to be useful aquaculture species. Attributes of ideal algal species as feed for aquaculture operations:
a. Must contain essential nutritive constituents.
b. Should be non toxic.
c. Rapid growth rates and amenable to mass culture.
d. Easily grow in cheap media.
e. Optimum size to be ingested by the feeding organisms: 1-15 μm for filter feeders and 10-100 μm for grazers.
f. Must be stable in culture to any fluctuations in temperature, light and nutrients as may occur in hatchery systems.
g. Readily digestible with an especially digestible cell wall.

The biochemical composition of microalgae, and therefore their nutritional value to fish and shellfish varies between species and is greatly affected by harvest stage, light intensity, nutrient concentrations, and culture methods (Brown et al., 1996, Otero and Fabregas, 1997). Further, it is known that the biochemical composition of algae can be altered by changing the growing conditions. Microalgae that have been found to have good nutritional properties—either as monospecies or within a mixed diet—include C. calcitrans, C. muelleri, P. lutheri, Isochrysis sp. (T.ISO), T. suecica, S. costatum and Thalassiosira pseudonana (Brown et al., 1997). Unsaturated fatty acids derived from microalgae, i.e. docosahexaenoic acid (DHA), eicosapentaenoic acid (EPA) and arachidonic acid (AA) are known to be essential for various larvae (Sargeant, et al., 1997). The fatty acid content showed systematic differences according to taxonomic group, although there were examples of significant differences between microalgae from the same class. Most microalgal species have moderate to high percentages of EPA. Prymnesiophytes (eg. Pavlova spp. and Isochrysis sp. (Isochrysis galbana, Tahiti; T ISO) and cryptomonads are relatively rich in DHA (0.2 to 11%), whereas eustigmatophytes (Nannochloropsis spp.) and diatoms have the highest percentages of AA (0 to 4%). Chlorophytes (Dunaliella spp. and Chlorella spp.) are deficient in both C20 and C22 PUFAs, although some species have small amounts of EPA (up to 3.2%). Because of this PUFA deficiency, chlorophytes generally have low nutritional value and are not suitable as a single species diet (Brown et al., 1997). Prasinophyte species contain significant proportions of C20 (Tetraselmis spp.) or C22 (Micromonas spp.)—but rarely both. Microalgae have an important role in aquaculture as a means of enriching zooplankton for on-feeding to fish and other larvae. In addition to providing protein (essential amino acids) and energy, they provide other key nutrients such as vitamins, essential PUFAs, pigments and sterols, which are transferred through the food chain. The species used in various aquacultural operations are listed in Table I (Adapted from De Pauw and Personne, 1988).

TABLE I
Microalgae species used in aquaculture industry

| | | | Extent of |
|---|---|---|---|
| | | | use in |
| Microalgae class and species | Nutritive value¹ | Used in² | industry³ |
| Bacillariophyceae | | | |
| Actinocyclus normanii | Epa | PL | + |
| Bellerochea polymorpha | Epa | BL, BP | + |
| Chaetocerso affinis | Epa, Vc, Sc | PL, BL, BP, AL | ++ |
| Chaetoceros calcitrans | Epa, Dha, Vc, Sc | PL, BL, BP, ML, BS, AL | +++ |
| Chaetoceros gracilis | Epa, Dha, Vc, Sc | PL, BL, BP, BS, AL, SC, MR | ++++ |
| Chaetoceros muellari | Epa, Dha, Vc, Sc | PL, BL, BP, BS, AL, SC | ++++ |
| Chaetoceros septentrionalis | Epa, Dha, Sc | PL, BP | + |
| Cylindrotheca closterium | Epa, Dha, Sc | PL, BS | +++ |
| Cyclotella nana | Epa, Aa | BS | +++ |
| Navicula pelliculosa | Epa, Dha | BS, BP | + |
| Nitzchia closterium | Epa, Dha | BS | +++ |
| Phaeodactylum tricornutum | Epa, Aa | PL, BL, BP, ML, BS, AL | ++++ |
| Skeletnema costatum | Epa, Aa, Sc | PL, BL, BP, BS, AL | ++++ |
| Thalassiospira pseudonana | Epa, Sc | PL, BL, BP, AL | ++++ |
| Chlorophyceae | | | |
| Brachiomonas submarina | Epa, Sc | PL | + |
| Chlamydomonas khaki | Epa, Sc | BS | + |
| Chlorella autotrohpica | Sc, Bc | BS | ++ |
| Chlorococcum spp. | Sc | BL, AL | + |
| Dunaliella tertiolecta | Sc, Bc, Aa Ax | PL, BS | ++ |
| Dunaliella salina | Sc, Bc, Ax | PL | ++ |
| Haematococcus pluvialis | Epa, Aa, Sc | PL, BS | ++ |
| Nannochloris atomus | Epa, Sc | PL, BL | + |
| Spongiococcum excentricum | Epa, Sc | PL | + |
| Tetraselmis chuii | Epa, Aa, Sc | BS | +++ |
| Tetraselmis suecica | Epa, Sc | PL, BL, BP | +++++ |
| Chryptophyceae | | | |
| Cryptomonas | Epa, Aa, Sc | PL, BL | + |
| Rhodomonas spp | Epa, Sc | PL, BP | ++ |
| Chroomonas salina | Epa, Sc | BP | ++ |
| Chrysophyceae | | | |
| Pyramimonas virginica | Dha, Epa | BP, BS | + |
| Micromonas pussila | Dha, Epa, Ax, Sc | BS | + |
| Cyanophyceae | | | |
| Spirulina platensis | Dha, Epa, Sc, Aa, Vc | BS, BP | + |
| Eustigmatophyceae | | | |
| Nannochloropsis oculata | Dha, Epa, Aa, Vc, Sc | PL, BL, BP, ML, BS, SC, FZ, MR | +++++ |
| Nannochloropsis gaditana | | | |
| Haptophyceae | Dha, Epa, Aa, Vc | PL, BL, BP, ML | ++++ |
| Coccolithus huxleyi | | | |
| Cricosphaera elongate | Dha, Epa | PL, BL | + |
| Dicrateria spp | Dha, Epa, Ax, Sc | BP, AL | + |
| Isochrysis galbana | Dha, Epa | BP, BS | + |
| Pavlova lutheri | Dha, Epa, Sc, Aa, Vc | PL, BL, BP, ML, BS, SC, FZ, MR | +++++ |
| Pavlova salina | Dha, Epa, Aa, Vc | PL BL, BP, ML, BS, MR | ++++ |
| Pseudoisochrysis paradoxa | Dha, Epa | PL, BL, BP, ML, BS | +++ |
| | Dha, Epa | PL, ML | + |

¹Dha—Docosahexanoic acid; Epa—Eicosapentanoic acid; Bc—Beta carotene: Ax—Astaxanthin; Aa—Arachidonic acid; Vc—Vitamin C; Sc—Saccharides.
²PL—penaeid shrimp larvae; BL—bivalve mollusk larvae; ML—freshwater prawn larvae; BP—bivalve mollusk post larvae; AL—abalone larvae; MR—marine rotifers; BS—brine shrimp; SC—saltwater copepods; FZ—freshwater zooplankton.
³+ Experimental and lab conditions; ++ moderately used; +++ widely used, ++++ most widely and commonly used; +++++ industry gold standard

Major aquacultural industrial suppliers are listed in Table II.

TABLE II
Major aquacultural industrial suppliers

| Product | Producer | Algal species |
|---|---|---|
| Phytoplex | Kent Marine | Nannochloropsis; Tetraselmis sp; Isochrysis sp |
| Phytomax | Kent Marine | Nannochloropsis; Tetraselmis sp; Isochrysis sp |
| Chromoplex | Kent Marine | Not available |
| Chromomax | Kent Marine | Not available |
| Phytoplan | Two Little Fishes | Arthrospira sp; Hematococcus sp; Schizochytrium; |
| | | Thalassiosira sp; |
| Marine show | Two Little Fishes | Isochrysis sp; Spirulina sp |
| Bioplankton | Liquid Life, USA | Nannochloropsis sp; Tetraselmis sp; Isochrysis sp |
| DT Live Marine | DT's Plankton Farm | Chlorella sp; Nannochloropsis oculata; |
| Phytoplankton | | Nannochloropsis salina |
| Phyto-Feast Live Marine | Reed Mariculture | Amphora; Isochrysis; Thalassiosira weissflogii; |
| | | Pavlova; Tetraselmis (20%); Nannochloropsis |
| Phyto-Feast Coral and Clam | Reed Mariculture | Amphora; Isochrysis; Thalassiosira weissflogii; |
| | | Pavlova; Tetraselmis (20%); Nannochloropsis |
| Instant Algae Nanno 3600 | Reed Mariculture | Nannochloropsis sp. |
| Shellfish Diet 1800 | Reed Mariculture | Isochrysis (25%); Thalassiosira weissflogii (30%); |
| | | Pavlova (20%); Tetraselmis (20%); |
| | | Nannochloropsis (5%) |
| IA-Spat Formula | Innovative Aqua | Tahitian isochrysis, Isochrysis galbana, Pavlova |
| | | lutheri, and Nannochloropsis oculata |
| IA-Starter Formula | Innovative Aqua | Chaetoceros-B, Phaeodactylum tricornutum; |
| | | Nannochloropsis oculata. |
| | | Nannochloropsis oculata; Phaeodactylum |
| | | tricornutum |
| IA-Enrichment Formula | Innovative Aqua | Chaetoceros-B, Phaeodactylum tricornutum |

Algae also can be used as a source of beta carotene and astaxanthin. Astaxanthin is one of a group of natural pigments known as carotenoids. In nature, carotenoids are produced principally by plants and their microscopic relatives, the microalgae. Animals cannot synthesize carotenoids de novo, thus ultimately they must obtain these pigments from the plants and algae that support their food chains. The primary use of synthetic astaxanthin today is as an animal feed additive to impart coloration to salmonids (salmon and trout), as well as to sea bream. Astaxanthin is a high-value carotenoid which is used as a pigmentation source in fish aquaculture. Besides the use of this compound as a colorant, it has been hypothesized that supplementation with astaxanthin might be a practical and beneficial strategy in management of human health due to its neuroprotective potential, its immunomodulating potential, and its antioxidant potential. The unicellular alga H. pluvialis is a suitable biological source for astaxanthin production. H. pluvialis is believed to be the world's richest known source of astaxanthin, a unique natural carotenoid pigment and biological antioxidant. When environmental conditions become adverse, i.e., when nutrients start becoming scarce, or the water pool starts drying out and the algae are increasingly exposed to direct sunlight, they enter a resting phase that allows them to survive for prolonged periods, until the environment becomes much more favorable. Today most astaxanthin is produced by total chemical synthesis and is sold at a price of $2,500 kg⁻¹; the high price and increasing demand for this compound as a food supplement provide a good opportunity for naturally produced astaxanthin.

Dunaliella salina and D. bardawil—the unicellular green microalgae (Chlorophyta, Dunaliellales)—are some of the richest sources of natural β-carotene, accumulating high levels of beta-carotene under growth-limiting conditions. When exposed to specific extreme environmental conditions, such as high light intensity, high salinity, extreme temperatures and/or nutrient deprivation, D. salina can accumulate up to 10% β-carotene of the dry algal biomass. This high carotene productivity has led to the large-scale application of D. salina and D. bardawil for the commercial production of natural β-carotene. Content of the high bioavailability stereoisomer of beta-carotene, the 9-cis stereoisomer, is highest in Dunaliella among all the natural carotenoids sources. The advantage of D. salina is that both production and accumulation of carotenoids in oil globules can be enhanced. Considering the recently developed transformation strategies for D. salina and its high carotenoid-accumulation capacity, this algal species might be a particularly suitable cell factory for the production of novel carotenoids. The major algal species used in various betacarotene and astaxanthin production using TWAG unit are listed in Table III.

TABLE III
Major algal species useful in betacarotene and astaxanthin production.

| Species | Product |
|---|---|
| Heamatococcus pluvialis | Astaxanthin |
| Dunaliella salina | betacarotene |
| Dunaliella tertiolecta | Betacarotene/Lutein |
| Dunaliella baldwifi | Betacarotene |
| Chlorella autotrophica | Betacarotene/zeaxanthin |
| Chorella vulgaris | Betacarotene |
| Spirulina platensis | Betacarotene |
| Arthrospria spp | Betacarotene |
| Osterearia | Phycobiliproteins |

Algae is also a source of highly unsaturated fatty acids. The benefits of long chain highly unsaturated fatty acids (HUFA i.e., fatty acids with 20 or more carbons and three or more double bonds) such as docasohexanoic acid (DHA, 22:6n-3), eicasapentanoic acid (EPA, 20:5n-3), arachidonic acid (AA, n-6), and gamma linolenic acid (GLA, 20:3n-3) in human health and development are well documented (Simopoulus, 1999; Connor, 2000). HUFA such as EPA and DHA exert a profound influence on the immune signaling pathways previously described for both humans and animals (Kelly and Daude 1993). Long-chain n-3 highly unsaturated fatty acids (HUFA) have beneficial effects on the cardiovascular system and reduce the severity of inflammatory diseases, arthritis, nephritis, lupus erythematosus, multiple sclerosis, strokes, cancer, skin diseases, asthma, and depression (Stansby 1990). Prostaglandins and leucotrienes constitute a group of extracellular mediator molecules that are part of an organism's defense system. Twenty-carbon HUFA are precursors of two groups of eicosanoids—prostaglandins and leucotrienes, with diverse pathophysiological actions including immune response and inflammatory processes. The major algal species which can be used in various highly unsaturated fatty acid productions using TWAG unit are listed in Table IV.

TABLE IV
Major algal species useful in highly unsaturated fatty acid production.

| Species | HUFA |
|---|---|
| Isochrysis galbana | Docahexanoic acid (22:6n-3) |
| Pavlova lutheri | |
| Pavlova salina | |
| Coccolithus huxleyi | |
| Dicrateria spp | |
| Cricosphaera elongate | |
| Pseudoisochrysis paradoxa | |
| Nannochloropsis oculata | |
| Nannochloropsis gaditana | |
| Micromonas pussila | |
| Pyramimonas virginica | |
| Thraustochytrium spp | |
| Crypthocodinium cohnii | |
| Amphidium spp. | |
| Schizochytrium spp. | |
| Skeletnema costatum | |
| Propyridium spp | |
| Ulkenia spp | |
| Nannochloris atomus | Eicosapentanoic acid (20:5, n-3) |
| Tetraselmis chuii | |
| Tetraselmis suecica | |
| Spongiococcum excentricum | |
| Monodus subterraneus | |
| Thalassiospira pseudonana | |
| Skeletnema costatum | |
| Phaeodactylum tricornutum | |
| Nitzchia closterium | |
| Navicula pelliculosa | |
| Cyclotella nana | |
| Cylindrotheca closterium | |
| Chaetoceros septentrionalis | |
| Chaetoceros muellari | |
| Chaetoceros gracilis | |
| Amphidium spp. | |
| Pavlova lutheri | |
| Pavlova salina | |
| Thraustochytrium spp | Arachidonic acid (20:4, n-6) |
| Parietochloris incisa | |
| Porphyridium cruentum | |

Algae are also highly suited as bioreactors for the large-scale production of foreign proteins for several reasons. First, they are relatively easy to culture as they will grow in a laboratory setting, subsisting on an inexpensive medium of simple salts. Second, unlike many cell lines, algae can be grown in continuous culture. Third, the cost for production on this platform was calculated to be approximately $0.002 per liter, compared to $1000-$2000 per gram in cultured mammalian cells and $0.05 per gram in a plant system. Besides the tremendous cost advantage, the generation of initial transformants to production volumes can occur within a short period of time. This system is also highly scalable in that transformed algal lines can be grown in few milliliters to 500,000 liters in a cost effective manner as their growth medium can be recycled. Furthermore, both the chloroplast and nuclear genome of algae can be genetically transformed, opening the possibility of expressing multiple recombinant products in a single organism. This eukaryotic system also offers the advantages of post-translational modifications of expressed protein products. The economics, ease of use, and flexibility of this system make it highly desirable for the expression of complex recombinant products. Over the last two decades, several highly efficient methods for nuclear, chloroplast and mitochondrial transformation have been developed for C. reinhardtii. Introduction of foreign DNA into the nuclear genome of C. reinhardii was initially performed using bombardment with DNA-coated microparticles, and/or agitation with glass beads or silicon carbide whiskers (Debuchy et al. 1989; Dunahay 1993; Gumpel and Purton 1994). Proteins, such as antibodies, in green algae is limited. It was not until 2003 when Mayfield et al. elegantly expressed human monoclonal antibodies in transgenic algal chloroplasts. In this work, C. reinhardtii chloroplast attpA or rbcL promoters were used to drive the expression of an engineered large single-chain antibody directed against herpes simplex virus (HSV) glycoprotein D. The antibody accumulated as a functional soluble protein in transgenic chloroplasts, and bound herpes virus proteins, as determined by ELISA assays. This breakthrough serves as the first demonstration of microalgae as an expression platform for complex recombinant proteins, and is currently being utilized by Rincon Pharmaceuticals Inc, a San Diego-based biopharmaceutical company, for expression of monoclonal antibodies for use in cancer therapy. The first report of successful manipulation of D. salina was by Geng et al. in 2003. Using electroporation, these investigators were able to generate stable transformants carrying the hepatitis B surface antigen. Walker et al. in 2005 reported the isolation and characterization of two D. tertiolecta nuclear RbcS genes and their corresponding 5′ and 3′ regulatory sequences. The functionality of these regulatory regions was initially used to drive the expression of a selectable marker in C. renhardtii. Subsequently, this expression cassette was electroporated into Dunaliella where both stable and transient transformants expressing the ble resistance gene were isolated. Jiang et al. (2005) identified and later used the 5′ flanking region of an actin gene from D. salina to direct stable expression of the bialaphos resistance gene (bar) in D. salina. The major algal species which can be used in various recombinant protein production using TWAG unit are listed in Table V.

TABLE V
Major algal species useful for recombinant protein production:

| Species | Seawater/Freshwater |
|---|---|
| Heamatococcus pluvialis | Seawater |
| Dunaliella salina | Seawater |
| Dunaliella tertiolecta | Seawater |
| Cyclotella nana | Seawater |
| Cylindrotheca closterium | Seawater |
| Chorella vulgaris | Seawater |
| Chorella autotrohica | Seawater |
| Phaeodactylum tricornutum | Seawater |
| Spirulina platensis | Seawater |
| Chlamydomonas reinharditii | Freshwater |
| Chlorella spp | Freshwater |

Exemplary recombinant protein products that can be obtained utilizing the methods and apparatus described herein include, but are not necessarily limited to: cytokines such as Interleukin-6 (IL-6), Interleukin-2 (IL-2), Interleukin-12 (IL-12), and Interleukin-4 (IL-4); antivirals such as griffithsin, cyanovirin, and PmAV; antibacterials such as lysozyme, melittin, moricin, cecropin, tachylepsin, defensin, and magainin; and bioactive peptides such as antifreeze protein; anti-inflammatory peptides, and anticoagulating proteins.

Renewable biofuels are needed to displace petroleum-derived transport fuels, which contribute to global warming and are of limited availability. Biodiesel, bioethanol and bio-H2 are potential fuels that have attracted the most attention. However, biodiesel and bioethanol produced from agricultural crops using existing methods cannot sustainably replace fossil-based transport fuels. Because of the high lipid content and fast growth, algae are considered to be one of the highly promising 3^{rd} generation sources of biofuel. The productivity of photosynthetic microbes in nature, on an aerial basis, exceeds that of terrestrial plants by approximately one order of magnitude. Also, biomass production need not compete with food production for either water or land. Both marine and freshwater can be used. Moreover, barren, arable (desert) land can be used. Renewable, carbon-neutral fuel application exploiting algal components include transesterification of lipids to biodiesel, saccharification of carbohydrates to ethanol, gasification of biomass to syngas, cracking of hydrocarbons and isoprenoids to gasoline and the direct synthesis of hydrogen gas. The major advantages of 3^{rd} generation microalgal systems are:
a. Have a higher photon conversion efficiency
b. Can be harvested batch-wise nearly all year round
c. Can utilize salt and waste water streams,thereby greatly reducing freshwater use
d. Can couple CO₂-neutral fuel production with CO₂ sequestration.
e. Produce non-toxic and highly biodegradable biofuels.
f. Costs associated with the harvesting and transportation of microalgae are relatively low compared to tree or terrestrial crops.
g. Algae can be grown under conditions which are unsuitable for conventional crop production.

The major algal species which can be used in various biofuel productions using WAG unit are listed in Table VI.

TABLE VI
Major Algal species useful for biofuel productions

| Species | Biofuel |
|---|---|
| Isochrysis galbana | Biodiesel |
| Pavlova lutheri | Biodiesel |
| Pavlova salina | Biodiesel |
| Coccolithus huxleyi | Biodiesel |
| Dicrateria spp | Biodiesel |
| Cricosphaera elongate | Biodiesel |
| Pseudoisochrysis paradoxa | Biodiesel |
| Nannochloropsis oculata | Biodiesel |
| Nannochloropsis gaditana | Biodiesel |
| Micromonas pussila | Biodiesel |
| Pyramimonas virginica | Biodiesel |
| Stichococcus spp | Biodiesel |
| Botryococcus braunii | Biodiesel |
| Nannochloris atomus | Biodiesel/Bio-ethanol |
| Tetraselmis chuii | Biodiesel/Bio-ethanol |
| Tetraselmis suecica | Biodiesel/Bio-ethanol |
| Spongiococcum excentricum | Biodiesel/Bio-ethanol |
| Thalassiospira pseudonana | Biodiesel/Bio-ethanol |
| Skeletnema costatum | Biodiesel/Bio-ethanol |
| Phaeodactylum tricornutum | Biodiesel/Bio-ethanol |
| Nitzchia closterium | Biodiesel/Bio-ethanol |
| Navicula pelliculosa | Biodiesel/Bio-ethanol |
| Cyclotella nana | Biodiesel/Bio-ethanol |
| Cylindrotheca closterium | Biodiesel/Bio-ethanol |
| Chaetoceros septentrionalis | Biodiesel/Bio-ethanol |
| Chaetoceros muellari | Biodiesel/Bio-ethanol/Bio-H₂ |
| Heamatococcus pluvialis | Biodiesel/Bio-ethanol/Bio-H₂ |
| Chaetoceros gracilis | Biodiesel/Bio-ethanol/Bio-H₂ |
| Chlorella vulgaris | Bio-H₂/Bio-ethanol |
| Chlorella autotrophica | Bio-H₂/Bio-ethanol/Bio-H₂ |
| Chlorella sorokiniana | Bio-H₂/Bio-ethanol/Bio-H₂ |
| Chlorella pyrenoidosa | Bio-H₂/Bio-ethanol/Bio-H₂ |
| Dunaliella salina | Bio-H₂/Bio-ethanol./Bio-H₂ |
| Dunaliella tertiolecta | Bio-H₂/Bio-ethanol |
| Chamydomonas reinharditii | Bio-H₂ |
| Spirulina platensis | Bio-H₂/Bio-ethanol |
| Amphora | Bio-H₂/Bio-ethanol/Biodiesel |
| Ettila oleoabundens | Bio-H₂/Bio-ethanol/Biodiesel |
| Ankistrodesmus falcatus | Bio-H₂/Bio-ethanol/Biodiesel |
| Nannochloris | Bio-H₂/Bio-ethanol/Biodiesel |
| Synechococcus spp | Bio-H₂/Bio-ethanol/Biodiesel |
| Synechocytis spp | Bio-H₂/Bio-ethanol/Biodiesel |
| Tribenema | Bio-H₂/Bio-ethanol/Biodiesel |
| Amphiprora hyalina | Bio-H₂/Bio-ethanol/Biodiesel |
| Crypthecodinium cohnii | Biodiesel |

Algae are also useful for nanotechnology applications. Nanotechnology is a fast-expanding area of science. Nanotechnology is the creation of useful materials, devices, and synthesis used to manipulate matter at an incredibly small scale between 1 and 100 nm, below the range of lithographic fabrication techniques (Lowe 2000; Whitesides, 2003). Nanometer-sized particles have novel optical, electronic, and structural properties that are not available either in individual molecules or bulk solids. The concept of nanoscale devices has led to the development of biodegradable self-assembled nanoparticles, which are being engineered for the targeted delivery of anticancer drugs and imaging contrast agents. Nanoconstructs such as these should serve as customizable, targeted drug delivery vehicles capable of ferrying large doses of chemotherapeutic agents or therapeutic genes into malignant cells while sparing healthy cells (Sinhi et al., 2006).

A key to the development of nanotechnology will be the ability to make complex nanoscaled three-dimensional structures at low cost and in large numbers. Diatoms are unicellular photosynthetic eukaryotes that are thought to contribute as much as 40% of marine primary productivity. A major component of the diatom cell wall is silica, which can account up to 50% of the dry weight of the cell, derived from silicon taken up from the environment (Levin and Guillard, 2003). Diatoms are the major silicifying organisms on the planet converting tons of soluble silicon into silica annually and most species have an obligate requirement for silicon (Si) for cell wall formation (Treguer et al., 1995). The wide variety of structures in the silicified cell walls of diatoms offers a promising natural source of such materials. Diatom silica can be converted into other materials, with maintenance of detailed morphology. To facilitate the use of diatoms in nanotechnology, specific manipulation of the structure in vivo will be desirable.

Silica becomes increasingly used in chemical, pharmaceutical, and nanotechnological processes, resulting in an increased demand for well-defined silicas and silica-based materials. The production of highly structured silica from cheap starting materials and under ambient conditions, which is a target for many researchers, is already realized in the formation of diatom biosilica, producing highly hierarchical ordered meso- and macropores silica structures. This notion formed the starting point for many research groups to study the integrative biomolecular and biomimetic mechanisms on diatom silicon biomineralization.

Because of the multiple uses of algae, there is a growing interest in the large-scale (high density) controlled-environment production of algae from different industries. To produce algae-derived materials at competitive prices, efficient large-scale growing units should be designed.

Some of the characteristics of units designed for large-scale production:
1. High production rates
2. Control contamination
3. Efficient re-cycling of the culture media
4. Amenable to scaling to very large scale production operations
5. Low external energy input
6. Efficient use of solar energy

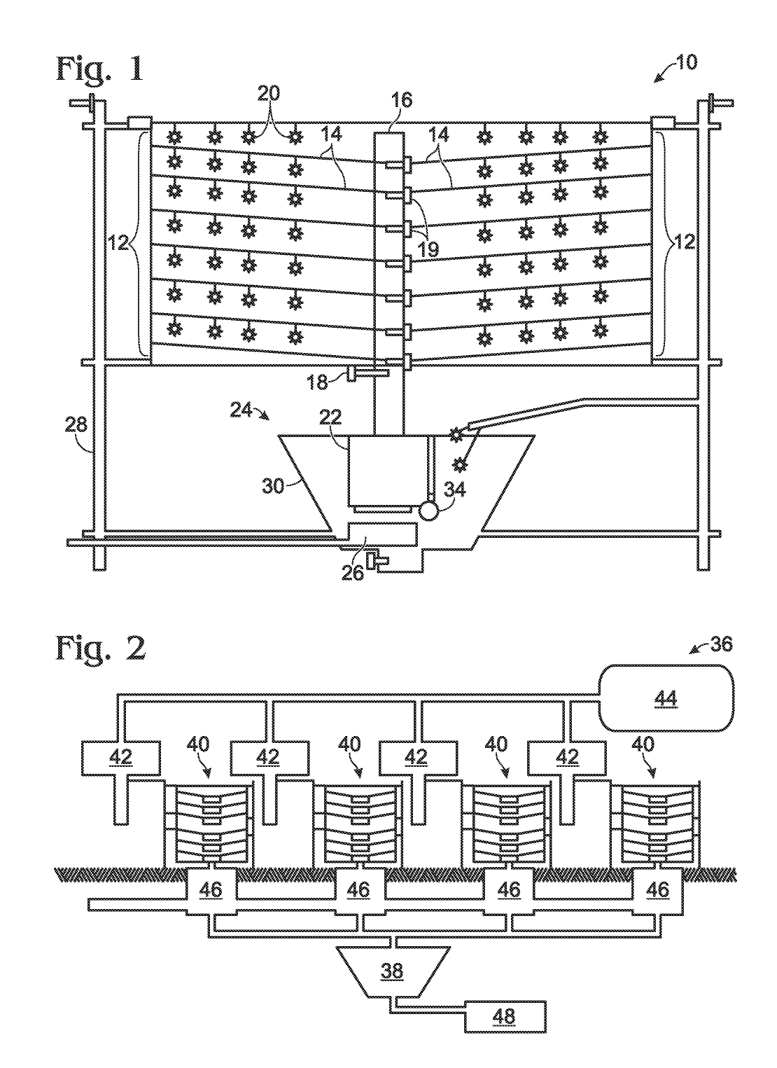

According to an embodiment, the present disclosure provides a terraced well alga growing (TWAG) unit. An exemplary embodiment is shown in FIG. 1. In general, the unit 10 is designed for optimum automation of multiple units arranged in arrays. The main algal unit is made of material with maximum light penetration such as, but not necessarily limited to, polyethylene, polycarbonate polypropylene, polyurethane, polyvinylpyrrolidone, polyvinylchloride, polystyrene, polybutylene, polyacrylate, or polyvinlyidene chloride. Each unit is sub-divided into a number of terraces 12. The panel of each terrace is made of sloping panels 14 that drain into a central channel 16 which is connected to an outlet via a valve 18. A series of valves 19 may control flow into the central channel. The top panel of each terrace has high water jet water outlets/showers 20. The outlet pipe which connects to all the chambers is connected to an algal sieve unit 22. According to a specific exemplary embodiment, the unit L×B×H=5m×5m×2 m and each unit is sub-divided into 7 terraces, each with a depth of 20 cm.

Once the algae is grown in the TWAG, the central channel 16 delivers the algae and culture water to a filtration unit including a collecting drum 26 where the algae is separated from the culture water via algal sieve 22. The culture water may then be recirculated to the growing unit via water transport system 28. The filtration unit may further include a sieve washing unit 32. The filtration unit will typically include suitable mechanical apparatus 34 for operating the sieve. The sieved algae is then collected in harvested algae collecting drum 30 and sent to a processing unit.

The TWAG may contain automated systems for maintaining the desired sun exposure, temperature, pH level, and culture mediums. For example, in order to maintain the desired working temperature, the unit may be configured such that temperatures above a desired range trigger the addition of cold seawater to the water bath until the desired reduction in temperature is achieved.

According to one exemplary embodiment, the TWAG unit is fully capable of utilizing energy from renewable sources such as solar, wind, and geothermal energy. For example, in a particular embodiment, the TWAG unit may be exposed to full sunlight, with average flux of at least 3000 μmol quantam⁻² s⁻¹ in summer The working temperature in this particular example is in the range of 20-32° C. The pH can maintained in the range of 7.3 to 7.8 by the addition of CO₂, until a desired endpoint is achieved. Suitable culture mediums include modified Bold's Basal medium, F/2 media (with or without silica) and/or artificial seawater. Table VII provides the specifications and Table VIII provides the production profile of an exemplary TWAG unit suitable for large scale algal growth.

TABLE VII
Specifications of individual TWAG unit

| Specifications | 5 × 5 × 2 | |
|---|---|---|
| Total volume | 50 | m³ |
| Width | 5 | m |
| Depth | 5 | m |
| Height | 2 | m |
| Thickness | 0.04 | m |
| Photon flux | 4000 | mol quanta m^{-2 s-1} |

TABLE VIII
Production profile of TWAG unit for biofuel production

| Variable | | |
|---|---|---|
| Oil content (%) | 45-50 | |
| Algae production (MT/h/yr) | 202.5 | |
| Algae production (g/m2/day) | 60-70 | g |
| Oil production (T/ha/yr) | 90-92 | |
| Oil production (bbl/ha/yr) | 800 | |
| Annual operating cost ($/ha) | 30,000 | |
| Production cost ($/bbl) | 40-45 | |

The present disclosure further provides a method of growing algae using the TWAG unit described herein. According to one embodiment, an exemplary method comprises initial inoculums of algal culture followed by a maximum cell growth phase. This is followed by a stressed cell growth phase, after which the algal cells can be harvested. The harvested cells can then be used to reinoculate other chambers in the unit. Exemplary protocols are provided below:

Initial Inoculum of algal culture—The inoculam of pure algal culture is added to the medium inlet valve in the inoculation port of the TWAG unit. Sufficient culture media is added via the overhead waterjet pumps inside the TWAG unit. At least 1×10⁴ algal cells/ml of media is optimum for the first inoculam. A dilution rate of 1:1-15 (inoculam:culture media) is recommended.

Maximum cell growth phase—In the first growth phase of the TWAG unit, the cells are grown in optimal growth media to attain high cell density for 24-36 hours. The list of species which can be used for TWAG operation are listed in Tables 1-4.

Stressed cell growth phase—Research on many strains demonstrated that, in general, nitrogen-sufficiency promoted high growth rates and low oil content, whereas nitrogen-deficiency reduced growth rates and resulted in high oil content (Lewin 1985). Silicon deficiency in diatoms yielded similar results; oil content accumulated to approximately 60% in a variety of culture trials. Oil content can be much higher than 40% —up to 59% in Stichococcus, and 86% in Botryococcus braunii. But such high values occur only after the cells have been stressed by nutrient deficiency and may no longer be dividing. In the second growth phase of the TWAG unit, the cells are put 24-30 h in nutrient deficient media to increase the oil content to the maximum before it is harvested. For this the media in the maximum growth phase is drained without the algal cell from the TWAG unit via a separate media draining channel. The TWAG is again filled with nutrient deficient media, the cell are properly mixed via water jet pump.

Harvesting—At the end of the growth phase-2, the algal cells are ready to harvest. The overhead water jet pump operates for 2 minutes to all the chambers to wash the attached algal cells and suspend all the algal material into slurry. The valves V1, V2 are all open simultaneously and algal culture will drain into the sieve-unit

Reinoculation—This step comes after the harvesting process. The algal culture in the C-1 unit is used as the inoculums for other chambers. The V2-V7 valves are closed, and the V1 valve is opened, so that algal culture in C-1 is now filled with the contents of the outlet tube from C-1.

According to a still further embodiment, multiple arrays of TWAG units may be used in the large farm-scale production of algae. For large scale production of algal biomass, arrays of TWAG unit are interconnected with simultaneous automated operation for all the connected TWAG units. Fully automated operation includes inoculation, growth phase, harvesting, and re-inoculation. As described herein, the harvested algae from the arrays of TWAG unit may be designed to drain into a single harvesting-sieve unit.

Table IX provides a comparison of features of traditional pond growing system, vertical tube system, and the presently described terraced well system.

TABLE IX
Growing systems comparison

| | | Traditional | Vertical | Terraced |
|---|---|---|---|---|
| | | pond | tube | Well |
| Features | | growing | system | Model |
| 1. | More algal production per unit | + | ++ | +++ |
| | area | | | |
| 2. | Controlled environment to | + | ++ | +++ |
| | reduce contamination | | | |
| 3. | Increased oil production via a | + | + | +++ |
| | two-step growth conditions | | | |
| 4. | Durable for longer periods | +++ | + | +++ |
| 5. | Amenable to very large | ++ | + | +++ |
| | production systems | | | |
| 6. | Water re-use | ++ | ++ | +++ |
| 7. | Evaporation control | + | +++ | +++ |
| 8. | Energy input for oil production | + | + | +++ |
| | process | | | |
| 9. | Automated production process | + | +++ | +++ |

According to an embodiment, two or more TWAGS may be arrayed together to increase production. FIG. 2 is a schematic side view of a TWAG array 36 comprising a common collection chamber 38. In this embodiment, the TWAG is designed to utilize gravity as a mechanism for encouraging fluid flow. As shown, each growing chamber 40 is fluidly connected to an overhead media tank 42 which provides water and nutrients to the growing algae. As shown, each media tank 42 may be fed from a common media tank 44. While not shown, but as described herein, common media tank 44 may receive recycled media obtained from various point along the herein described processes. Each growing chamber is also fluidly connected to a below ground collection tank 46, which is then fluidly connected to a lower common collection chamber 38, which is, itself, connected to a grinder 48.

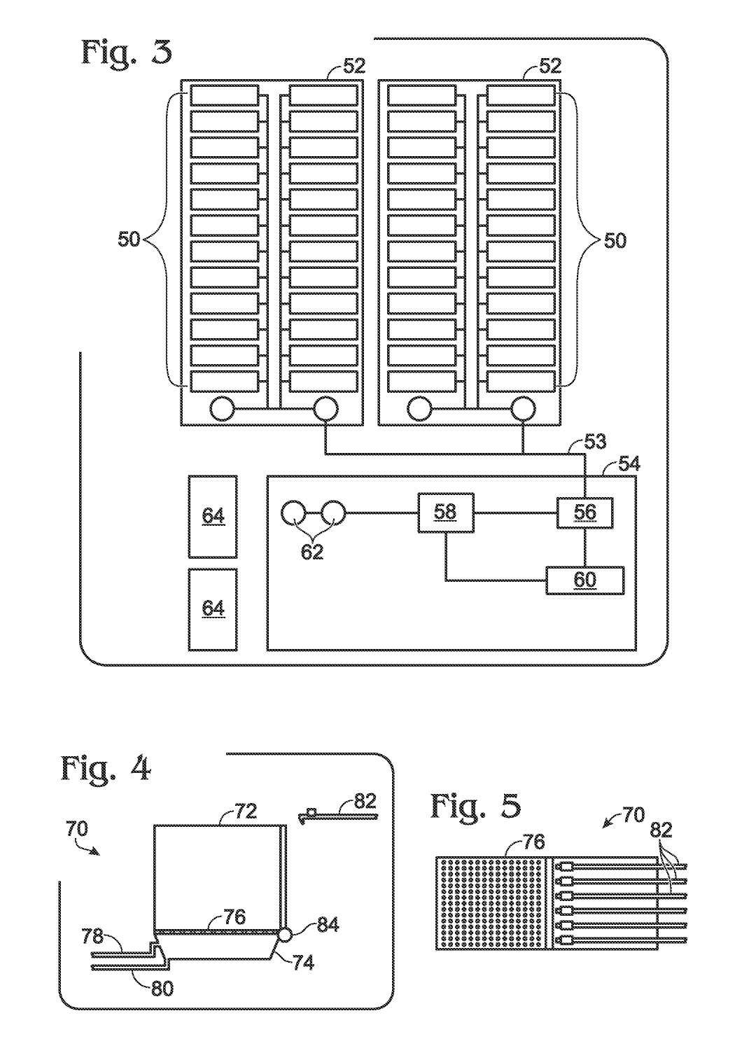

FIG. 3 is a schematic overhead view of 48 individual TWAG units 50 arrayed in two separate green houses 52. As shown, the cultured algal product from each greenhouse is collected and sent via transport line 53 to a separate facility 54, which may, for example, include sequential oil extraction apparatus 56, oil purification apparatus 58, feed collection apparatus 60, and biodiesel reactor stations 62. Biodiesel storage vessels 64 may be located nearby.

It should be appreciated that filtration plays an important role in the sequential processing of algae. Accordingly, the present disclosure provides for a filtration unit which is configured to separate the algae from the culture media in which it is grown, producing an isolated algal mass and reusable culture media. Typically, algae will be grown in water that may or may not contain additional nutrients. For the purposes of the present disclosure “culture water” will be used to describe a water-based culture media in which algae is grown that may or may not contain additional nutrients or substances. When referring to the reuse or recycling of the culture water, the culture water may or may not undergo additional processing such as purifying before it is returned to the system.

The filtration unit will typically include some type of separation device, such as a sieve or screen, means for removing the separated algal mass from the filtration unit, and means for washing the separation device. In the embodiment described below, a sieve is configured to rotate in order to remove the algal mass from the filtration unit. However, it will be appreciated that any other suitable apparatus could be employed including removal by hand, the use of an automated scraper or shovel, vibration, conveyer belts or the like. Similarly, in the embodiment described below the means for washing the separation device comprises a high jet spray, however other suitable systems could be employed that may include high compression air jets, brushes, or the like.

FIGS. 4-11 are schematic representations of an exemplary algal filtration unit 70. As perhaps best seen in FIGS. 4 and 5, the filtration unit includes a harvesting chamber 72 which is fluidly connected to a culture water collection chamber 74. A sieve 76 separates the harvesting and culture water collection chambers. The culture water collection chamber further includes a vacuum unit 78 and a culture water drainage tube 80, which as described above, may recycle the culture water back to the growing unit(s). A plurality of high jet sieve washers 82 also forms part of the unit. The harvesting chamber further includes a mechanical rotor 84 configured to move the sieve.

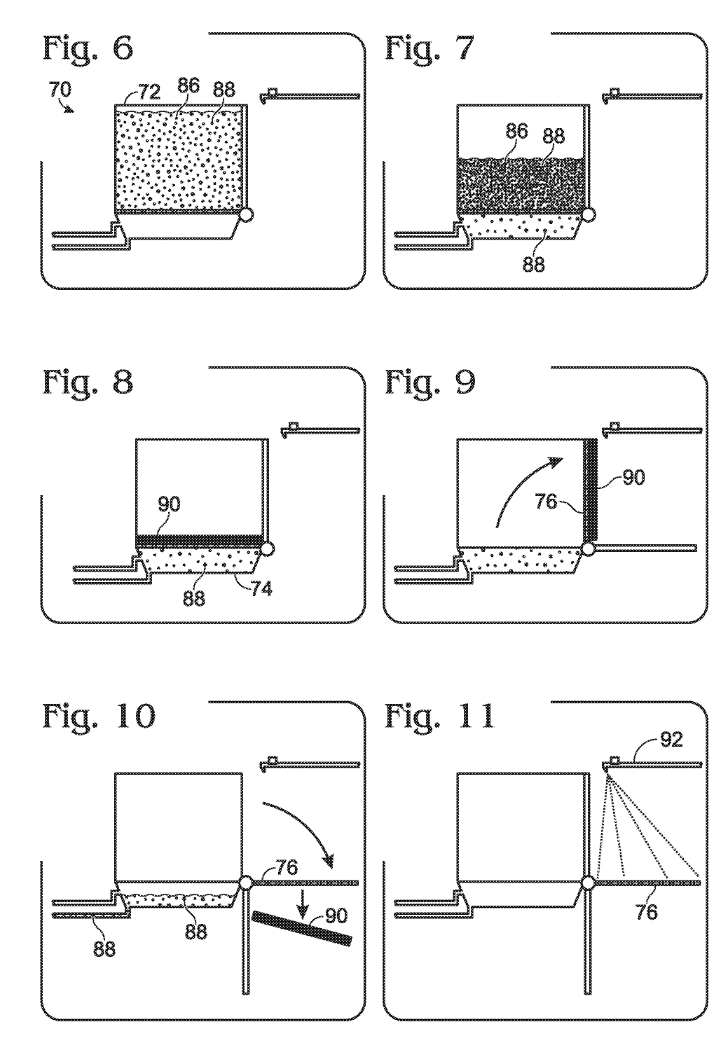

Operation of the algal filtration unit is best understood when viewing FIGS. 6-11. Turning first to FIG. 6, the algae 86 and culture water 88 in which it is grown is directed to the harvesting chamber 72 of the filtration unit. As shown in FIG. 7, the culture water is then pulled through the sieve and into the culture water collection chamber by use of the vacuum pump, leaving an algal biomass 90 in the harvesting chamber, as shown in FIG. 8. The culture water 88 in chamber 74 can then be recycled as described above or otherwise disposed of. Turning now to FIG. 9, the sieve 76 is mechanically shifted 90 degrees, removing the drained algal biomass from the harvesting chamber. At FIG. 10, the sieve is mechanically shifted a further 90 degrees (or 180 degrees from the original orientation) to permit the algal biomass to be collected and moved to further processing. As shown at FIG. 11, the high jet sieve washer 92 then washes the sieve. The washed sieve unit is then returned to its original orientation and ready for another round of batch filtration.

According to yet another embodiment of the invention, once produced, the algal biomass is sequentially processed for recombinant proteins, highly unsaturated fatty acids (DHA, EPA, AA and GLA), and biodiesel. The structured processing of the algal biomass through these various sequential steps produces many valuable products from the biomass which will substantially increase the unit value of the raw material. The high market demand of the two by-products, algal meal and glycerin, of the sequential processing, can be integrated into the value chain. Accordingly, the combined value of the products and byproducts decreases the operational costs and significantly increase the operational profit. Exemplary species which can be sequentially processed using the herein described techniques are listed above in Tables I-VI.

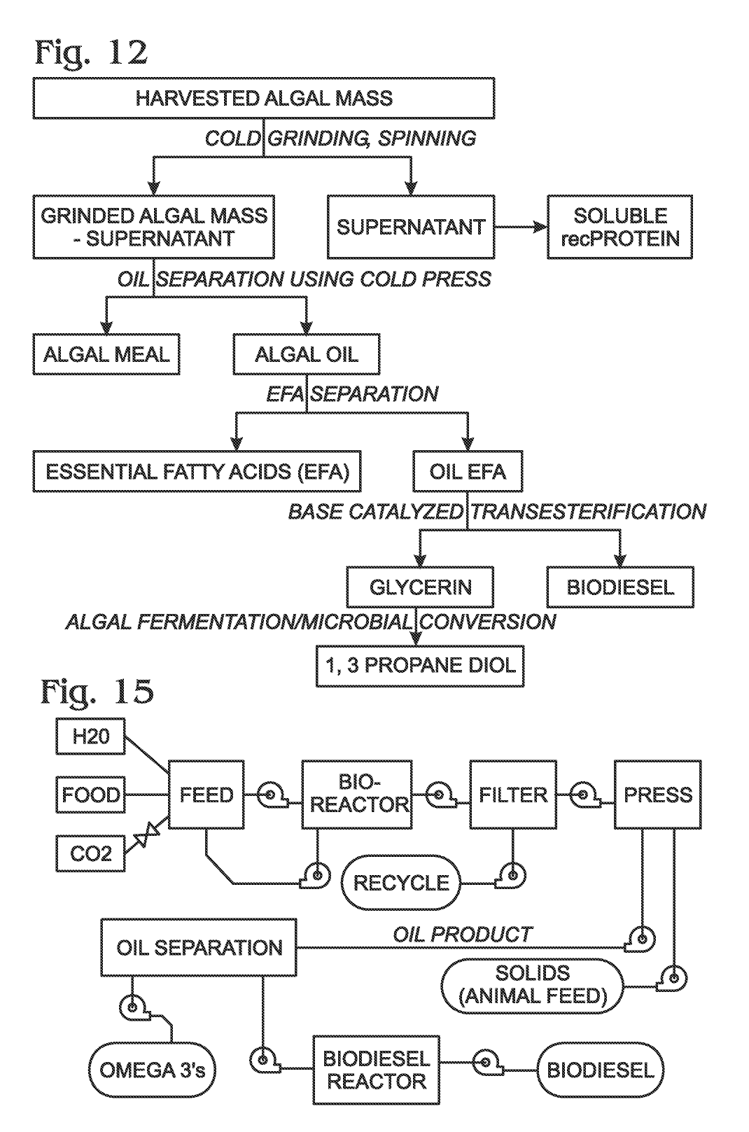

A flowchart depicting an example of sequential processing of the algae is shown in FIG. 12. As shown, harvested algal mass is processed via cold grinding and spinning to produce ground algal mass+supernatant and supernatant. The supernatant can be further processed to produce soluble recombinant proteins. The ground algal mass+supernatant can be processed to separate the algal oil from the algal meal using, for example, a cold press. The algal oil can then undergo essential fatty acid (EFA) separation to produce essential fatty acids and EFA-containing oil. The EFA-containing oil can then be processed using, for example, base catalyzed transesterification, to produce glycerin and biodiesel. Glycerin can be processed, for example via algal fermentation and microbial conversion, to produce 1,3 propane diol. As explained above, each of these products, recombinant proteins, algal meal, EFAs, glycerin, biodiesel, and 1,3 propane diol are desirable and can be sold separately in order to produce a wide variety of revenue streams.

Table 6 provides a list of products that can be derived from the herein-described process. FIG. 6 displays the estimated value of these products.

TABLE 6
Integrated sequential processing and production system: value chain

| PRODUCT | EXAMPLE | VALUE | MARKET |
|---|---|---|---|
| Rec-Proteins | Cytokines-Interleukin-6 (IL-6), IL-2, IL- | High | Pharmaceutical industry |
| | 12, IL-4) | | Diagnostic industry |
| | Interferon gamma and beta | | |
| | Antivirals - Griffithsin, Cyanovirin, | High | Pharmaceutical industry |
| | PmAV | | |
| | Antibacterials - lysozyme, melittin, | | Pharmaceutical industry |
| | moricin, cecropin, tachylepsin, defensin, | | |
| | magainin | | |
| | Bioactive peptides - Antifreeze protein | | Pharmaceutical industry |
| | Anti-inflammatory peptides | | Pharmaceutical industry |
| | Anticoagulating proteins | | Pharmaceutical industry |
| | Neutralizing antibodies, single chain | | |
| | antibodies against multiple viral and | | |
| | bacterial infections | | |
| Fatty acids | Docasahexanoic acid | High | Nutritional food industry |
| | Eicosapentanoic acid | Moderate | Feed industry |
| | Alpha linolenic acid | Moderate | |
| | Arachidonic acid | Moderate | |
| Carotenoids | Astaxantin, Zeaxanthin, Lutein | High | Feed |
| | | | Nutraceutical industry |
| Fluorescent label | Phycoerythrin, Phycocyanin | High | Biomedical research |
| Biofuel | Biodiesel, Bioethanol, Biohydrogen, | High | Transportation industry |
| | Aviation fuel | | |
| Meal | Algal meal-containing residual amounts | Moderate | Feed industry (poultry, fish, |
| | of docasahexanoic acid and | | shrimp) |
| | eicosapentanoic acid | | |
| By-product | Glycerin | Low | Biodiesel industry, Algal |
| | | | farms |
| Chemicals | Agar, Alginin, Caragennin, | Moderate | Biotechnology and food |
| | | | industry |
| Industrial Bulk | Ethylene, Propylene, caprolactam | Moderate | Chemical Industry |
| Chemicals | 1,3 propanediol | | |
| Nanotech-devices | Finished silicon, Silicon nanochips from | High value | Lithography, |
| | Diatoms, | | Semiconductors, Nanotech |
| | | | industries |

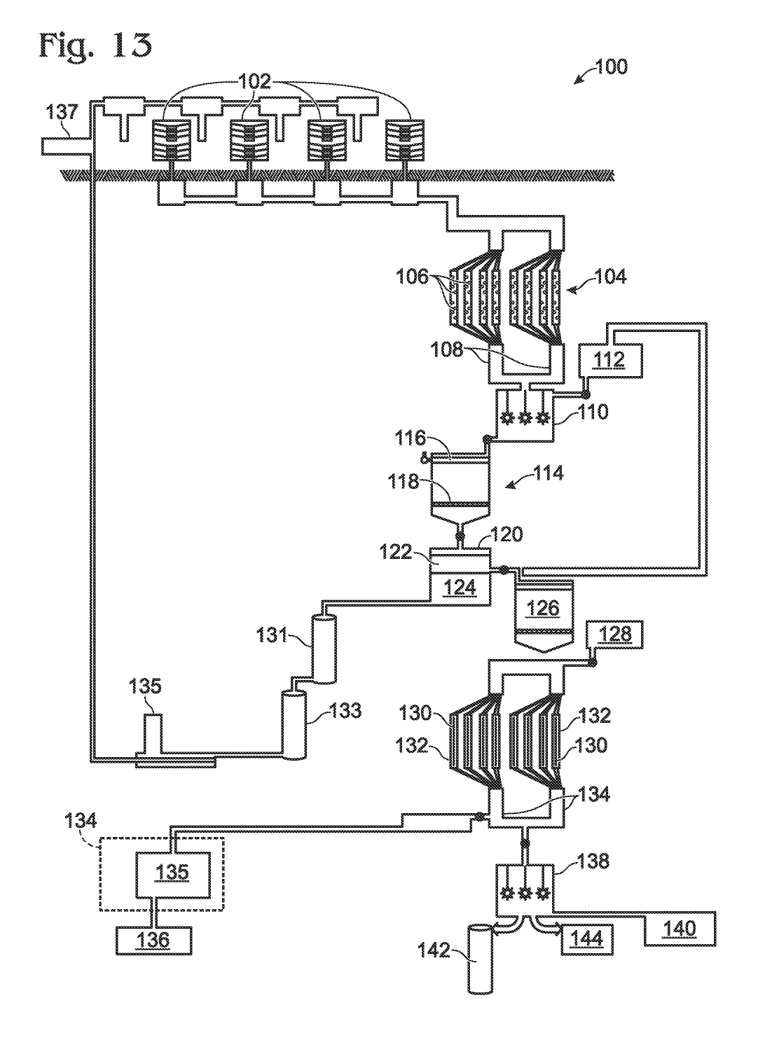

FIG. 13 is a schematic representation of an exemplary facility 100 configured to grow and sequentially process algal biomass. In the depicted embodiment, the facility comprises a plurality of TWAG units 102. Drained algal mass from the growing units is directed to sonication tubes 104 which may include, for example, a plurality of individual sonication probes 106. The sonicated algal mass is then collected in collection channels 108 and directed towards a solvent mixing tank 110, which is fed solvent from a solvent tank 112. Suitable solvents include but are not limited to hexane, isohexane, acetone, and ethyl acetate. The algae-solvent mixture is then introduced into a filtration unit 114 including a mechanical press 116 and an algal particulate sieve 118. A phase separation tank 120 is configured to separate the liquid into a hexane-lipid layer 122 and a water layer 124. The hexane-lipid layer is then introduced into a hexane evaporation and condensation unit 126. A methanol tank 128 feeds methanol to the crude lipid exiting the hexane evaporation and condensation unit and the resulting mixture is subjected to immobilized lipase columns 130 inside of transesterification columns 132. Collection channels receive the resulting fatty acid methyl esters. From here, the glycerin is removed and transferred to a 1,3 propanediol production unit 134 which may use, for example, algal/microbial fermentation tank 135 to produce 1,3 propanediol, which may be stored in tank 136. Meanwhile, the fatty acid methyl esters are collected in tank 138, from which the biodiesel is removed and stored in biodiesel storage tank 140. A portion of the fatty acid methyl esters may be sent to column chromatography unit 142, for example, in order to purify the highly unsaturated fatty acids while another portion is subjected to urea complexation and filtration in tank 144 in order to remove the highly unsaturated fatty acids.

After separation in the phase separation tank, the water layer is sent first through a recombinant-protein affinity column 131 bearing a first antibody and then a second recombinant protein affinity column 133 bearing a second antibody. The water is then sent to a UV sterilization unit 135 and then back to the nutrient replenishment tank 137 for recycling through the system.

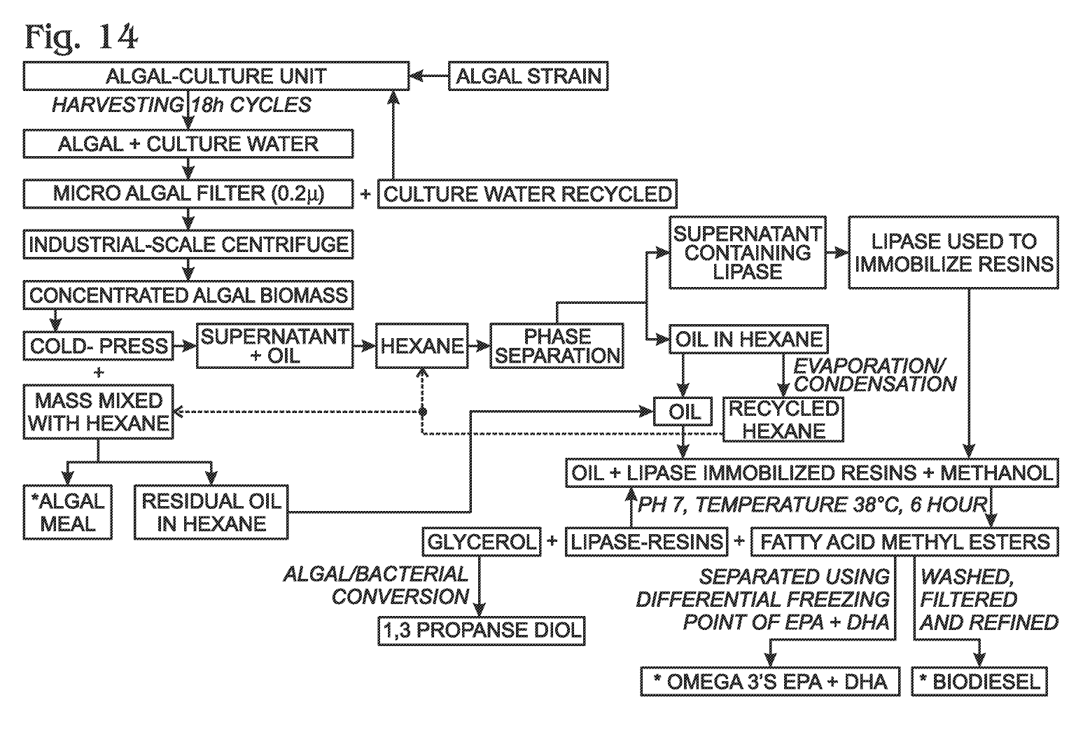

Turning now to FIG. 14, a flowchart showing another exemplary sequential processing method is shown. Initially a desired algal strain is identified. A typical desired algal strain has a high growth rate, expresses methanol tolerant lipase, and has a high DHA and EPA content. Examples include, but are not limited to, Chlorella vulgaris+Phaedactylum tricarnutum (30%+70% seedling). Next, the algal strain is introduced to an algal-culture unit. A typical algal culture unit may be sized to contain about 4-5 g/L of algae and liquid nutrients. According to a specific exemplary embodiment, the atmosphere may comprise about 2-4% flue gas, 800-1200 ppm Nitrogen, 400-500 ppm phosphorus, sodium nitrate, glucose, and have a culture pH of between 7 and 7.5. Multiple harvesting cycles then result in a product of algal+culture water which is then filtered, for example using an 0.2μ filter, thereby resulting in an algal mass and culture water. The culture water can then be recycled back to the algal-culture unit. The algal mass is then subjected to an industrial-scale centrifuge in order to concentrate the algal biomass. The concentrated algal biomass is then introduced to a cold press to remove the supernatant and oil. The algal mass is then mixed with hexane to produce algal meal and residual oil in hexane. Separately, the supernatant and oil is mixed with hexane and subjected to phase separation to produce oil in hexane, which is then evaporated and condensed, and supernatant containing lipase. The lipase is then used to immobilize resins. The oil in hexane from both hexane steps is then separated to obtain hexane (which can then be recycled back into the system) and oil. The oil is mixed with the lipase immobilized resins and methanol to obtain fatty acid methyl esters, lipase-resins, and glycerol. The fatty acid methyl esters can be separated using differential freezing points, for example, to obtain various fatty acids such as Omega 3's, EPA and DHA. Moreover, the fatty acid methyl esters can also be washed, filtered, and refined to produce biodiesel. Using algal/bacterial conversion, the glycerol is then converted to ⅓ propanediol.

FIG. 15 is a block flow diagram showing yet another embodiment of a sequential process. Initially, water, carbon dioxide and food are fed to growing algae which are then introduced to a bioreactor, and then to a filter. The waste water from the filter is then recycled back into the system. After filtration, the algae are introduced into a press to produce both solid and oil products. The solid product (i.e. algal mass) can be used as animal feed while the oil products are subjected to oil separation to ultimately obtain Omega 3s and biodiesel.

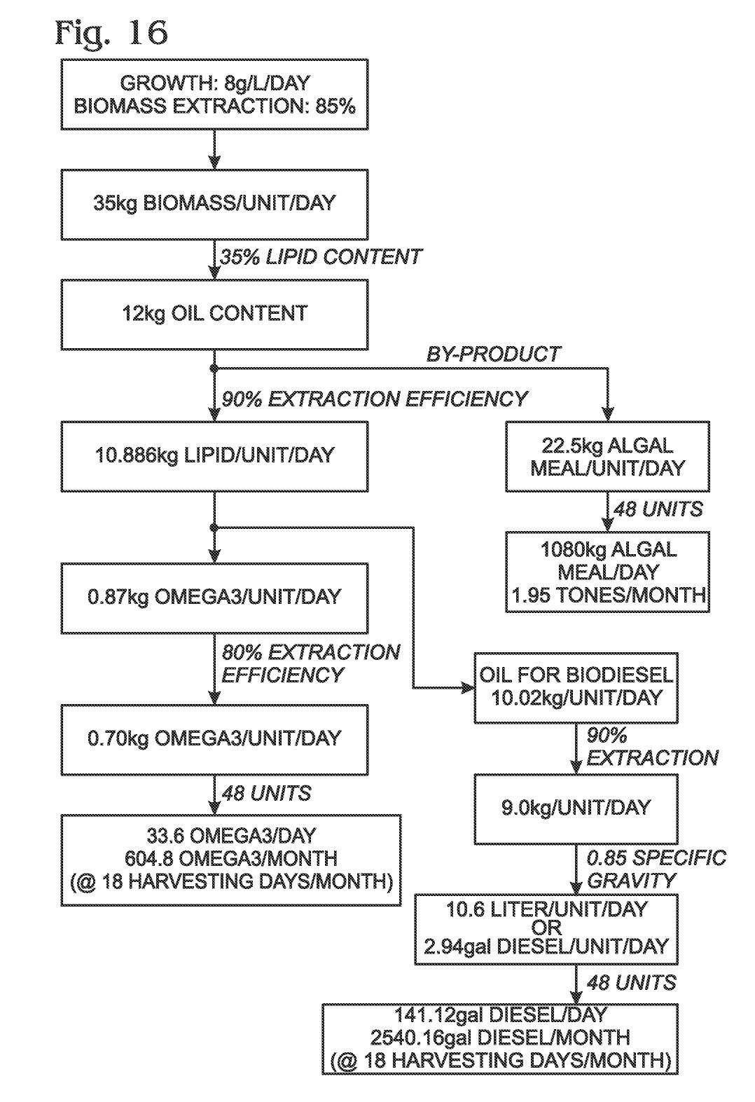

FIG. 16 is a flowchart showing exemplary production yields when practicing the methods described herein.

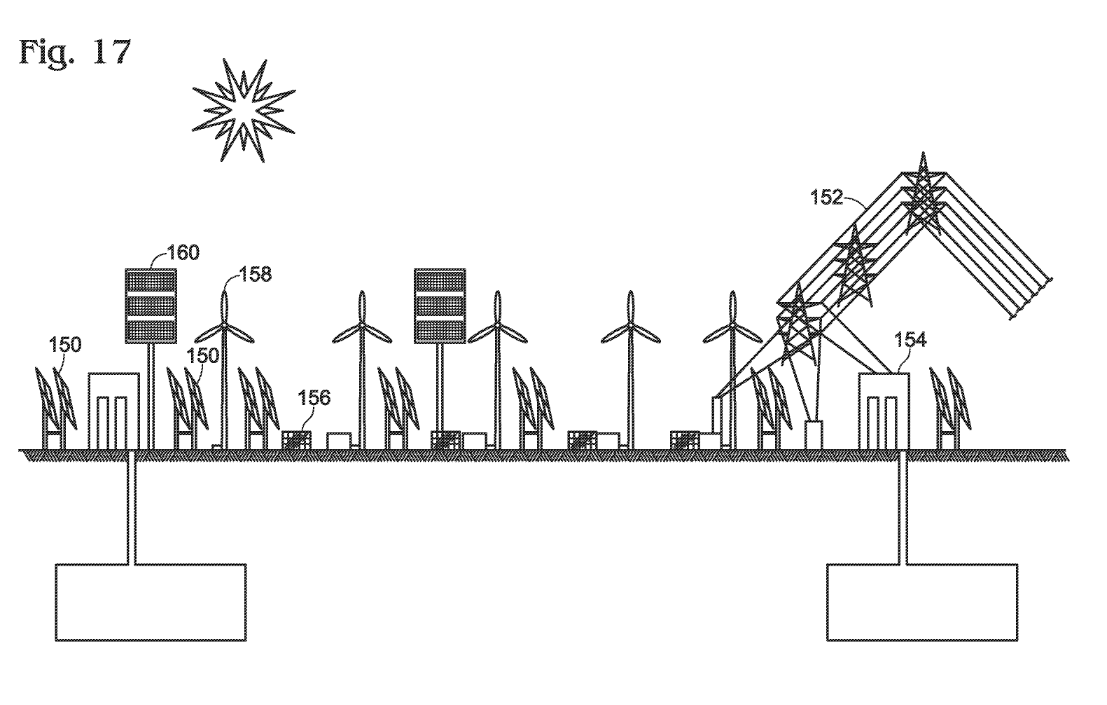

According to an embodiment, the presently described methods and apparatus utilize a wide variety of renewable energy sources, as shown in FIG. 17. For example, solar panels 150 could be used to obtain solar energy which could be used to both to create electricity f152 controlled by a smart green grid 154 or running the machinery as well as to heat the greenhouses 156. Wind energy obtained from windmills 158 or other similar systems could further be used to produce renewable electricity. Similarly, carbon capture devices 160 could be utilized to obtain carbon dioxide for algal growth. Placement of some or much of the processing systems underground could allow the system to rely on geothermal sources of renewable electricity as well as heat energy for algal processing and extraction of liquid fuel. Furthermore, as described throughout the disclosure various products and byproducts can be recycled and reused throughout the system, significantly reducing waste, energy consumption, and overall costs.

The following references are incorporated by reference:

Brown, M. R. The amino acid and sugar composition of 16 species of microalgae used in mariculture. Journal of Experimental Marine Biology and Ecology, 145:75-99.

Brown, M. R., Dunstan, G. A., Jeffrey, S. W., Volkman, J. K., Barrett, S. M. and LeRoi, J. M. (1993a). The influence of irradiance on the biochemical composition of the prymnesiophyte Isochrysis sp. (clone T-ISO). Journal of Psychology, 29: 601-612.

Brown, M. R., Garland, C. D., Jeffrey, S. W., Jameson, I. D., Leroi, J. M., 1993b. The gross and amino acid compositions of batch and semi-continuous cultures of Isochrysis sp. (clone T.ISO), Pavlova lutheri and Nannochloropsis oculata. Journal of Applied Psychology, 5: 285-296.

Brown, M. R., G. A. Dunstan, S. J. Norwood & K. A. Miller. 1996. Effects of harvest stage and light on the biochemical composition of the diatom Thalassiosira pseudonana. J. Phycol. 32:64-73.

Brown, M. R., Jeffrey, S. W., Volkman, J. K., Dunstan, G. A., 1997. Nutritional properties of microalgae for mariculture. Aquaculture, 151: 315-331.

Brown, M. R. 2002. Nutritional value of microalgae for aquaculture. In: Cruz-Suarez, L. E., Ricque-Marie, D., Tapia-salazar, M., Gaxiola-Cortes, M. G. Simoes, N (Eds). Avances en Nutricion Acuicola VI. Memorias del VI Simposium Internacional de Nutricion Acuicola. 3 al 6 de Septiembre de; 2002. Cancun, Quintana Roo. Mexico.

Daume, S. 2003. Early life history of abalone (Haliotis rubra, H. laevigata): settlement, survival and early growth. Final report for FRDC project 1998/306. Department of Fisheries. Western Australia. Fisheries Research Contract Reports 3:1-110.

Daume, S., B. M. Long & P. Crouch. 2003. Changes in amino acid content of an algal feed species (Navicula sp.) and their effect on growth and survival of juvenile abalone (Haliotis rubra). J. Appl. Phycol. 15:201-207.

De Pauw, N. and Persoone, G. 1988. Micro-algae for aquaculture. In: Micro-algal Biotechnology. Borowitzka, M. A. and L. J. Borowitzka (Eds.). Cambridge University Press, Cambridge, U.K., pp 197-221.

Domínguez, A., Ferreira, M., Coutinho, P., Fábregas, J., Otero, A. 2005. Delivery of astaxanthin from Haematococcus pluvialis to the aquaculture food chain. Aquaculture 250:424-30.

Enright, C. T., Newkirk, G. F. Craigie, J. S., Castell, J. D., 1986b. Evaluation of phytoplankton as diets for juvenile Ostrea edulis L. Journal of Experimental Marine Biology and Ecology, 96: 1-13.

Enright, C. T., Newkirk, G. F., Craigie, J. S., Castell, J. D., 1986a. Growth of juvenile Ostrea edulis L. fe Chaetoceros calcitrans Schütt of varied chemical composition Journal of Experimental Marine Biology and Ecology, 96: 15-26.

Fabregas, J., A. Otero, E. Morales, B. Cordero & M. Patino. 1996. Tetraselmis suecica cultured in different nutrient concentrations varies in nutritional value to Artemia. Aquaculture 143:197-204.

Fabregas, J., A. Otero, E. Morales, B. O. Arredondo-Vega & M. Patino. 1998. Modification of the nutritive value of Phaedactylum tricornutum for Artemia sp. in semicontinuous culture. Aquaculture 169:167-176.

Fabregas, J., Herrero, C., 1986. Marine microalgae as a potential source of minerals in fish diets. Aquaculture, 51: 237-243.

Fábregas, J., Otero, A., Dominguez, A., Patiño, M. 2001. Growth rate of the microalga Tetraselmis suecica changes the biochemical composition of Artemia species. Marine Biotechnology 3:256-263

Gara, B., Shields, R. J., McEvoy, L., 1998. Feeding strategies to achieve correct metamorphosis of Atlantic halibut, Hippoglossus hippoglossus L., using enriched Artemia. Aquaculture Research, 29: 935-948.

Harrison, P. J., Thompson, P. A., Calderwood, G. S., 1990. Effects of nutrient and light limitation on the biochemical composition of phytoplankton. Journal of Applied Psychology 2: 45-56.

Knauer, J., and Southgate, P. C. 1999. The review of nutritional requirments of bivalves and the development of alternative and artificial diets for bivalve aquaculture. Reviews in Fisheries Science, 7: 241-280.

Knuckey, R. M., Brown, M. R., Barrett, S. M., Hallegraeff, G. M., 2002. Isolation of new nanoplanktonic diatom strains and their evaluation as diets for the juvenile Pacific oyster (Crassostrea gigas). Aquaculture 211:253-274.

Kreeger, D. A. and C. J. Langdon. Effect of dietary protein content on growth of juvenile mussels, Mytilus trossulus (Gould 1850). Biological Bulletin 185: 123-139

Merchie, G., Lavens, P., Dhert, Ph., Dehasque, M., Nelis, H., De Leenheer, A., Sorgeloos, P., 1995. Variation of ascorbic acid content in different live food organisms. Aquaculture, 134: 325-337.

Nichols, P. D., Holdsworth, D. G., Volkman, J. K., Daintith, M., Allanson, S., 1989. High incorporation of essential fatty acids by the rotifer Brachionus plicatilis fed on the prymnesiophyte alga Pavlova lutheri. Australian Journal of Marine and Freshwater Research, 40: 645-655.

Otero, A., Fabregas, J. 1997 Changes in the nutrient composition of Tetraselmis suecica cultured semicontinuously with different nutrient concentrations and renewal rates. Aquaculture 159:111-123

Otero, A., Fábregas, J. 1997 Changes in the nutrient composition of Tetraselmis suecica cultured semicontinuously with different nutrient concentrations and renewal rates. Aquaculture 159:111-123.

Parsons, T. R., Stephens, K., Strickland, J. D. H. 1961. On the biochemical composition of eleven species of marine phytoplankters. Journal of Fisheries Research Board Canada, 18: 1001-1016.

Preetha, R Jayaprakash, N S Bright Singh I S Synechocystis MCCB 114 and 115 as putative probionts for Penaeus monodon post-larvae. Dis Aquat Organ. 2007 74 (3):243-247

Renaud, S. M., Thinh, L. V. Parry, D. L., 1999. The gross composition and fatty acid composition of 18 species of tropical Australian microalgae for possible use in mariculture. Aquaculture, 170: 147-159

Roberts, R. D., T. Kawamura & C. M. Nicholson. 1999. Growth and survival of post-larval abalone (Haliotis iris) in relation to development and diatom diet. J. Shellfish Res. 18:243-250.

Rodríguez, C., Perez, J. A., Badía, P., Izquierdo, M. S., Fernández-Palacios, H., Lorenzo Hernández, A., 1998. The n-3 highly unsaturated fatty acid requirements of gilthead seabream (Sparus aurata L.) larvae when using an appropriate DHA/EPA ratio in the diet. Aquaculture 169: 9-23. Parsons, T. R., Stephens, K., Strickland, J. D. H., 1961. On the chemical composition of eleven species of marine phytoplankters. Journal of the Fisheries Research Board Canada, 18: 1001-1016.

Rønnestad, I, Helland, S., Lie, O., 1998. Feeding Anemia to larvae of Atlantic halibut (Hippoglossus hippoglossus L.) results in lower larval vitamin A content compared with feeding copepods. Aquaculture, 165: 159-164.

Sargent, J. R., McEvoy, L. A., Bell., J. G., 1997. Requirements, presentation and sources of polyunsaturated fatty acids in marine fish larval feeds. Aquaculture, 155: 117-127.

Thompson, P. A., Guo, M.-X., Harrison, P. J., 1993. The influence of irradiance on the biochemical composition of three phytoplankton species and their nutritional value for larvae of the Pacific oyster (Crassostrea gigas). Marine Biology, 117: 259-268.

Watson, D., S. Daume, J. Prince, L. Beasley & B. Knott. 2004. The influence of culture conditions on the growth and biochemical composition of algal feed species for juvenile greenlip abalone (Haliotis laevigata). Molluscan Research 25 (1), 1-8.

Webb, K. L., Chu, F. E., 1983. Phytoplankton as a food source for bivalve larvae. In: G. D. Pruder, C. J. Langdon and D. E. Conklin (Editors), Proceedings of the Second International Conference on Aquaculture Nutrition: Biochemical and Physiological Approaches to Shellfish Nutrition, Louisiana State University, Baton Rouge, La. pp. 272-291.

Whyte, J. N. C., Bourne, N., Hodgson, C. A., 1989. Influence of algal diets on biochemical composition and energy reserves in Patinopecten yessoensis (Jay) larvae. Aquaculture, 78: 333-347.

Wikfors, G. H., Ohno, M., 2001. Impact of algal research in aquaculture. Journal of Physiology, 37: 968-974

Chapter (algal nanotechno)

Alivisatos A. Perspectives on the physical chemistry of semiconductor nanocrystals. J Phys Chem 1996; 100:13226-39.

Azam, F. Silicic acid uptake in diatoms studied with [68Ge] Germanic acid as a tracer. Planta 121, 205 (1974).

Azam, F. Hemmsingsen, B. B and. Volcani, B. E Role of silicon in diatom metabolism V. Silicic acid transport and metabolism in the heterotrophic diatom Nitzschia alba. Arch. Microbiol. 97, 103 (1974).

Bhattacharyya P. and Volcani, B. E. Sodium dependent silicate transport in the apochlorotic marine diatom Nitzschia alba. Proc. Natl. Acad. Sci. 77, 6386 (1980).

Belton D J, Patwardhan S V, Annenkov V V, Danilovtseva E N, Perry C C. From biosilicification to tailored materials: optimizing hydrophobic domains and resistance to protonation of polyamines. PNAS 2008 105(16):5963-5968

Davis, M. E. Ordered porous materials for emerging applications. Nature 417, 813 (2002).

Del Amo Y. and Brzezinski, M. A. The chemical form of dissolved Si taken up my marine diatoms. J. Phycol. 35, 1162 (1999).

Drum R W, Gordon R. 2003. Star Trek replicators and diatom nanotechnology. Trends Biotechnol. 21(8), 325-328.

Falciatore A, Bowler C. 2002 Revealing the molecular secrets of marine diatoms. Annu Rev Plant Biol. 53,109-130.

Fei B, Hu Z, Lu H, Xin J H. 2007. Preparation of a panoscopic mimic diatom from a silicon compound. Small. 3(11): 1921-1926.

Frigeri L G, Radabaugh T R, Haynes P A, Hildebrand M. 2006. Identification of proteins from a cell wall fraction of the diatom Thalassiosira pseudonana: insights into silica structure formation. Mol Cell Proteomics. 5(1), 182-193.

Gordon R, Kling H J, Sterrenburg F A. 2005. A guide to the diatom literature for diatom nanotechnologists. J Nanosci Nanotechnol. 5(1), 175-178.

Gordon R, Parkinson J. 2005. Potential roles for diatomists in nanotechnology. J Nanosci Nanotechnol. 5(1), 35-40.

Grachev M A, Annenkov V V, Likhoshway Y V. Silicon nanotechnologies of pigmented heterokonts. Bioessays. 2008 30:328-37.

Hildebrand M. 2005. Prospects of manipulating diatom silica nanostructure. J Nanosci Nanotechnol. 5(1), 146-157.

Hildebrand, M Volcani, B. E. Gassmann, W. and. Schroeder , J. I A gene family of silicon transporters. Nature 385, 688 (1997).

Hildebrand, M. Dahlin, K and Volcani, B. E. Characterization of a silicon transporter gene family in Cylindrotheca fusiformis: Sequences, expression analysis, and identification of homologs in other diatoms. Mol. Gen. Genet. 260, 480 (1998).

Hildebrand, M. Higgins, D. R. Busser, K and Volcani, B. E. Silicon-responsive cDNA clones isolated from the marine diatom Cylindrotheca fusiformis. Gene 132, 213 (1993). J. Phycol. 21, 168 (1985).

Kröger , N. Deutzmann, R. and Sumper , M. Polycationic peptides from diatom biosilica that direct silica nanosphere formation. Science 286, 1129 (1999).

Kröger, N. Deutzmann, R. Bergsdorf, C and Sumper, M. Species specific polyamines from diatoms control silica morphology. Proc. Natl. Acad. Sci. 97, 14 133 (2000).

Kröger, N. Lorenz, S Brunner, E. and. Sumper, M Self assembly of highly phosphorylated silaffins and their function in biosilica morphogenesis. Science 298, 584 (2002).

Kröger N. 2007. Prescribing diatom morphology: toward genetic engineering of biological nanomaterials. Curr Opin Chem Biol. 11(6), 662-669.

Lewin J. C. and Guillard, R. R. L. Diatoms. Annu. Rev. Microbiol. 17, 373 (1963).

Losic D, Rosengarten G, Mitchell J G, Voelcker N H. 2006. Pore architecture of diatom frustules: potential nanostructured membranes for molecular and particle separations. J Nanosci Nanotechnol. 6(4), 982-989.

Lowe, C. R. Nanobiotechnology: the fabrication and applications of chemical and biological nanostructures. Current Opinion in Structural Biology 10, 2000: 428-434

Manning T J, Purcell J, Nienow J A, Olsen E, Riddle K, Ekman J. 2005. Comparison of diatoms, exfoliated graphite, single-wall nanotubes, multiwall nanotubes, and silica for the synthesis of the nanomagnet Mn12. J Nanosci Nanotechnol. 5(1), 167-174.

Mayama S, Kuriyama A. Diversity of mineral cell coverings and their formation processes: a review focused on the siliceous cell coverings. J Plant Res. 2002 August; 115(4):289-95. Epub 2002 Jun. 19.

Mock T, Samanta M P, Iverson V, Berthiaume C, Robison M, Holtermann K, Durkin C, Bondurant S S, Richmond K, Rodesch M, Kailas T, Huttlin E L, Cerrina F, Sussman M R, Armbrust E V. 2008. Whole-genome expression profiling of the marine diatom Thalassiosira pseudonana identifies genes involved in silicon bioprocesses. Proc Natl Acad Sci USA. 105(5):1579-1584.

Pappas J L. 2005 Geometry and topology of diatom shape and surface morphogenesis for use in applications of nanotechnology. J Nanosci Nanotechnol. 5(1),120-130.

Poulsen N, Sumper M, Kröger N. 2003. Biosilica formation in diatoms: characterization of native silaffin-2 and its role in silica morphogenesis. Proc Natl Acad Sci USA. 100(21),12075-12080.

Poulsen, N. Sumper, M and. Kröger, N Biosilica formation in diatoms: Characterization of native siliffan-2 and its role in silica morphogenesis. Proc. Natl. Acad. Sci. 100, 12 075 (2003).

Reidel G. F and. Nelson, D. M Silicon uptake by algae with no known Si requirement. II. Strong pH dependence of uptake kinetic parameters in Phaeodactylum tricornutum (Bacillariophyceae).

Sinha, R Gloria J. Kim, Shuming Nie and Dong M. Shin Nanotechnology in cancer therapeutics: bioconjugated nanoparticles for drug delivery Mol Cancer Ther. 2006; 5:1909-1917.

Sandhage, K. H. Dickerson, M. B Huseman, P. M. Caranna, M. A. Clifton, J. D. Bull, T. A. Heibel, T. J Overton, W. R. Schoenwaelder, M. E. A. Novel, bioclastic route to self-assembled, 3D,

chemically tailored meso/nanostructures: Shape-preserving reactive conversion biosilica (diatoms) microshells. Adv. Mater.14, 429 (2002).

Sumper M, Brunner E, Lehmann G. 2005. Biomineralization in diatoms: characterization of novel polyamines associated with silica. FEBS Lett. 579(17), 3765

Suntherland A. Quantum dots as luminescent probes in biological systems. Curr Opin Solid State Mater Sci 2002; 6:36-370.

Thamatrakoln K, Hildebrand M. 2005. Approaches for functional characterization of diatom silicic acid transporters. J Nanosci Nanotechnol. 5(1),158-166.

Tréguer, T. Nelson, D. M. Van Bennekom, A. J. Leynaert, A and Queguiner, B. The silica balance in the world ocean: A reestimate. Science 268, 375 (1995).

Umemura K, Liao X, Mayama S, Gad M. 2007. Controlled nanoporous structures of a marine diatom. J Nanosci Nanotechnol. 7(8),2842-2846.

Unocic R R, Zalar F M, Sarosi P M, Cai Y, Sandhage K H. 2004. Anatase assemblies from algae: coupling biological self-assembly of 3-D nanoparticle structures with synthetic reaction chemistry. Chem Commun (Camb). 7(7), 796-797.

Vrieling E G, Sun Q, Beelen T P, Hazelaar S, Gieskes W W, van Santen R A, Sommerdijk N A. 2005. Controlled silica synthesis inspired by diatom silicon biomineralization. J Nanosci Nanotechnol. 5(1), 68-78.

Vrieling, E. G C., Gieskes W. W., and Beelen, T. P. M. Silicon deposition in diatoms: Control by the pH inside the silicon deposition vesicle. J. Phycol. 35, 548 (1999).

Wee K M, Rogers T N, Altan B S, Hackney S A, Hamm C .2005. Engineering and medical applications of diatoms. J Nanosci Nanotechnol. 5(1), 88-91.

Whitesides, G. M. The Right Size: Nanobiotechnology” Nature Biotech., 2003, 21, 1161-116

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications. The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising:
growing cultured algae in culture water in an algae growing unit,
harvesting the algae,
supplying the harvested algae to a filtration unit comprising:
a harvesting chamber comprising a sieve having pores sized to maintain algae within the harvesting chamber while allowing culture water to exit the harvesting chamber;
apparatus for forcing the culture water through the sieve and into a culture water reservoir thereby leaving an algal biomass in the harvesting chamber; and
apparatus for removing the algal biomass from the harvesting chamber once the culture water has exited the harvesting chamber;
forcing the culture water to exit the harvesting chamber thereby leaving an algal biomass in the harvesting chamber;
removing the algal biomass from the harvesting chamber once the culture water has exited the harvesting chamber;
suspending the algal biomass in a solvent;
separating the suspended algal biomass into a water phase and an oil phase; and
processing the oil phase to obtain biofuels.

2. The method of claim 1 wherein the algae growing unit comprises a plurality of sloping terraces configured to drain into a central channel; wherein each terrace is supplied with culture water from a common culture water reservoir; and wherein the central channel communicates with the filtration unit and the method of supplying the harvested algae to a filtration unit comprises transporting the algae via the central channel.

3. The method of claim 2 further comprising processing the oil phase to obtain unsaturated fatty acids

4. The method of claim 2 further comprising processing the oil phase to obtain algal meal.

5. The method of claim 2, further comprising processing the oil phase to obtain oil.

6. The method of claim 2 wherein the cultured algae and algal culture water contains secreted or unsecreted recombinant proteins or peptides, the method further comprising isolating the recombinant proteins or peptides.

7. The method of claim 2 further comprising recycling the culture water back to the algal growing unit.

8. The method of claim 2 further comprising processing the oil phase to obtain glycerin.

9. The method of claim 8 further comprising processing the glycerin to obtain 1,3 propanediol or 1,2 propanediol.

10. A facility for sequentially processing alga to produce a variety of products, the facility comprising:
an algae growing unit configured to grow cultured algae in culture water;
a filtration unit configured to separate the cultured algae from the culture water, the filtration unit comprising:
a harvesting chamber comprising a sieve having pores sized to maintain algae within the harvesting chamber while allowing culture water to exit the harvesting chamber;
apparatus for forcing the culture water through the sieve and into a culture water reservoir thereby leaving an algal biomass in the harvesting chamber; and
apparatus for removing the algal biomass from the harvesting chamber once the culture water has exited the harvesting chamber;
a solvent unit configured to suspend the separated cultured algae in a solvent;
a phase separation unit configured to separate the suspended algae into an oil phase and a water phase;
an oil product processing unit configured to produce glycerin, biodiesel, and highly unsaturated fatty acids from the oil phase.

11. The facility of claim 10 further comprising a recombinant protein separation unit configured to produce recombinant proteins from the algal water phase and algal culture water.

12. The facility of claim 10 wherein the algae growing unit is heated, at least in part, via solar energy.

13. The facility of claim 10 wherein the filtration unit is configured to recirculate the separated culture water back to the algae growing unit.

14. The facility of claim 10 wherein the oil product processing unit is configured to purify a solvent and recirculate the purified solvent back to the solvent unit.

15. The facility of claim 10 wherein the algae growing unit comprises a plurality of sloping terraces configured to drain into a central channel; wherein each terrace is supplied with culture water from a common culture water reservoir; and wherein the central channel communicates with the filtration unit.

16. (canceled)

17. (canceled)

18. (canceled)

19. (canceled)

20. The facility of claim 17 wherein a saline aquifer is used as a culture water resource.

21. A filtration unit for processing of algae grown in culture water, the filtration unit comprising:
a harvesting chamber comprising a sieve having pores sized to maintain algae within the harvesting chamber while allowing culture water to exit the harvesting chamber;
apparatus for forcing the culture water through the sieve and into a culture water reservoir thereby leaving an algal biomass in the harvesting chamber; and
apparatus for removing the algal biomass from the harvesting chamber once the culture water has exited the harvesting chamber.

22. The filtration unit of claim 21 further comprising a washing apparatus configured to wash the sieve after the algal biomass has been removed from the harvesting chamber.

23. (canceled)

24. The filtration unit of claim 21 wherein the apparatus for forcing the culture water through the sieve comprises a vacuum pump.

25. The filtration unit of claim 21 wherein the apparatus for forcing the culture water through the sieve comprises a mechanical press.

26. (canceled)
